(19) United States
(12) Patent Application Publication
(10) Pub. No.: US 2014/0030714 A1
(43) Pub. Date: Jan. 30, 2014

(54) Method and means for distinguishing malignant from benign tumor samples, in particular in routine air dried fine needle aspiration biopsy (FNAB)

(71) Applicants: Ralf Paschke, Leipzig (DE); Markus Eszlinger, Markranstadt (DE)

(72) Inventors: Ralf Paschke, Leipzig (DE); Markus Eszlinger, Markranstadt (DE)

(73) Assignee: UNIVERSITÄT LEIPZIG, Leipzig (DE)

(21) Appl. No.: 14/008,557

(22) PCT Filed: Mar. 28, 2012
(86) PCT No.: PCT/EP2012/055567

§ 371 Date: Sep. 29, 2013

Related U.S. Application Data
(60) Provisional application No. 61/488,172, filed on May 20, 2011.

(30) Foreign Application Priority Data
Mar. 30, 2011 (EP) 11160484.9

(51) Int. Cl. C12Q 1/68

(52) U.S. Cl. CPC C12Q 1/6886
USPC 435/6.11; 435/6.14; 435/6.12; 536/24.1

(57) ABSTRACT

The invention concerns a method and means for distinguishing malignant from benign tumor samples of the thyroid, by performing a RNA extraction in a standard fine needle aspiration biopsy (FNAB) sample, in particular an air dried FNAB smear. The presence of gene-rearrangements and/or the expression of miRNA is analyzed in the isolated RNA, wherein the presence of a gene-rearrangement and/or the differential expression of miRNA is indicative for a malignant tumor.

Method and means for distinguishing malignant from benign tumor samples, in particular in routine air dried fine needle aspiration biopsy (FNAB)

The invention concerns a method for distinguishing malignant from benign tumors in routine samples, in particular in routine air dried fine needle aspiration biopsy (FNAB) in particular of thyroid nodules.

Thyroid nodules are frequent clinical findings. Their reported prevalence varies from 3-76% depending on the screening method and the population evaluated. However, the incidence of thyroid cancer is low. The annual incidence in areas not affected by nuclear fall out has been reported to range between 1.2-2.6 cases per 100.000 in men and 2.0-3.8 cases per 100,000 in women with higher incidences in countries like Sweden, France, Japan and USA. Therefore, patients with thyroid nodules require evidence based strategies for the differential diagnosis and risk stratification for malignancy.

Fine-needle aspiration biopsy (FNAB) is the most sensitive and specific tool for the differential diagnosis of thyroid malignancy. Some limitations of FNAB can be overcome by the molecular analysis of FNAB. The preoperative FNAB can reduce the number of surgeries for thyroid nodules to 10% as compared to a strategy without FNAB use with a concomitant increase of thyroid malignancies from 3.1 (without FNAB) to 34% (with FNAB). Under optimal conditions, 60-80% of the biopsied nodules can be classified as benign by cytology and 3.5-5% are classified as malignant. However, the ratio of malignant/benign results for patients undergoing resection of thyroid nodules is still 1:15 in Germany or 1:7 in Italy thus resulting in a high number of “diagnostic” thyroid surgeries (among the 110,000 annual thyroid surgeries in Germany). Besides other reasons this unsatisfactory situation is mainly due to some limitations of this FNAB focused strategy like the difficulty to determine the rate of false negative cytologies since a nodule diagnosed as benign by FNAB is usually managed conservatively and especially because 10-20% of the FNAB samples are classified as follicular proliferation/indeterminate which cannot distinguish between follicular adenoma (FA), adenomatoid hyperplasia (AH), follicular thyroid carcinoma (FTC), and follicular variant of papillary thyroid carcinoma (fvPTC). Therefore, patients with this cytologic finding currently have to undergo (diagnostic) surgery, which will detect thyroid malignancy in about 20% of these patients. This means that 80% of the thyroid FNAB samples that were classified as follicular proliferation/indeterminate lesion by cytology will undergo diagnostic (unnecessary) thyroidectomy. Thus, the follicular proliferation category is the most problematic FNAB category.

In recent years immunohistologic markers like Galectin-3, HBME-1, Fibronectin-1, CITED-1, Cytokeratin-19 have been investigated in order to improve the differential diagnosis between benign and malignant thyroid nodules. However, they have barely been adopted in daily routine diagnostics, mainly because of different methods used and because these markers show prominent overlap between FA and differentiated thyroid carcinomas. Currently, no single cytochemical (or genetic) marker is specific and sensitive enough to reliably further specify or replace the morphologic diagnosis of follicular lesion or suspicious for neoplasm.

U.S. Pat. No. 7,319,011 B2 describes that Follicular thyroid adenoma (FTA) can be distinguished from follicular thyroid carcinoma (FTC) by comparing amount of an expression product of at least one gene selected from the group consisting of DDIT3, ARG2, ITM1, C1orf24, TARSH, and ACO1 in a test follicular thyroid specimen to a normal control.

U.S. Pat. No. 7,670,775 B2 discloses methods of identifying malignant thyroid tissue comprising testing a thyroid tissue sample for the expression of at least two genes chosen from CCND2, PCSK2, and PLAB.

U.S. Pat. No. 6,723,506 B2 describes the molecular characterization of PAX8-PPAR1 molecules for detection and treatment of certain tumors, particularly thyroid follicular carcinomas.

U.S. Pat. No. 7,378,233 B2 describes the T1796A mutation of the BRAF gene that was detected in 24 (69%) of the papillary thyroid carcinomas examined. Further, the T1796A mutation was detected in four lung cancers and in six head and neck cancers but not in bladder, cervical, or prostate cancers.

MicroRNAs (miRNAs or miR) are short ribonucleic acid molecules that are post-transcriptional regulators that bind to complementary sequences on target messenger RNA transcripts (mRNAs), usually resulting in translational repression and gene silencing. miR-221 is known to be up-regulated in PTC (Pallante P et al. 2006. Endocr Relat Cancer 13(2):497-508; He H et al. 2005 PNAS 102(52):19075-19080).

With the discovery of somatic mutations for 42% of PTCs and 65% of FTCs new perspectives for the classification and diagnosis of thyroid tumors in addition to histology have emerged. Molecular testing for somatic mutation has become an immediate and currently the most promising future approach for molecular FNAB diagnosis which will allow a further discrimination of the follicular proliferation/indeterminate and suspicious FNAB categories, to reduce the number of diagnostic thyroid surgeries and the rate of false negative cytologies. Nearly all of the somatic mutations have been tested for their applicability in FNAB diagnosis in different settings in the recent years. Most studies have analyzed one mutation e.g. BRAF or RET/PTC (e.g. Cheung C. C. et al. 2001. J Clin Endocrinol Metab 86(5):2187-2190). Four studies have analyzed several mutations (BRAF, RAS, RET/PTC and PAX8/PPARg) (Moses W et al. 2010 World J Surg 34(11):2589-2594; Cantara S et al. 2010 J Clin Endocrinol Metab 95(3):1365-1369; Nikiforov Y E et al. 2009 J Clin Endocrinol Metab 94(6):2092-2098; Ohori N P et al. 2010 Cancer Cytopathol 118(1):17-23).

Most studies analyzing one or more mutations (especially the studies investigating the rearrangements) in FNAB material use a separate part of the same FNAB sample fresh or after storage in frozen form for cytology and molecular analysis (e.g. left over cells in the needle bevel plus the needle washing, or a third or part of the total FNAB material obtained). Others do not use the same FNAB sample for both morphologic and molecular analyses but performed an additional FNAB which can increase the likelihood of generating contradictory results even more. Most important, all of these strategies require to collect fresh extra material, mostly by additional needle sticks from all patients undergoing FNA to then only use this additional material for the 20% of the patients with the cytology diagnosis “indeterminate” or “follicular proliferation”.

Only some studies that investigated only BRAF with genomic DNA extraction used the FNAB material from routine air dried or ethanol fixed FNAB smears (e.g. Girlando S, et al. 2010 Int J Surg Pathol 18(3):173-176 and Kim J W et al. 2010. J. Clin. Endocrinol. Metab. 95(8): 3693-3700).

Extraction of RNA from air dried stained cells of cytology FNAB smears for the RT-PCR amplification of RET/PTC and PAX8/PPARG rearrangements has up to date not been reported and has been judged to be not feasible (Nikiforova M N, Nikiforov Y E 2009, Thyroid 19(12):1351-1361).

Thus a goal of the invention is to provide a method for distinguishing malignant from benign tumor samples to better facilitate tumor diagnosis in routine samples and to further reduce the number of diagnostic surgeries and the rate of false negative cytologies.

This goal is solved by the invention in a first aspect by providing a method for distinguishing malignant from benign tumor samples, in particular of the thyroid, by
a.) performing a RNA extraction in an air dried and/or fixed fine needle aspiration biopsy (FNAB) sample,
b.) analyzing the presence of gene-rearrangements and/or differential expression of miRNA in the isolated RNA, wherein the presence of a gene-rearrangement and/or the differential expression of miRNA is indicative for a malignant tumor.

Preferably, in step a.) RNA, including miRNA, and DNA are extracted from the air dried and/or fixed FNAB sample simultaneously. The extraction of the nucleic acids is preferably performed from a routine stained FNAB sample used for routine cytology. Preferably miRNA is extracted quantitatively from the FNAB sample.

Preferably, in step b.) differential expression of miRNA is analyzed. Thereby, a differential expression of miRNA is indicative for a malignant tumor and/or used to distinguish malignant and benign tumors.

The method according to the invention provides the following advantages:
It is possible, to perform a cytology analysis and the method according to the invention (including RNA extraction) in exactly the same sample. Thereby, inconsistent results resulting from divergent samples are avoided. Accordingly, the method according to the invention allows a direct correlation between cytology and molecular analysis.
The method according to the invention avoids additional needle sticks to obtain fresh extra material for (potential) molecular diagnosis from all patients undergoing FNA.
For FNAB samples which do not give a clear diagnosis based on cytology criteria, integrated and focussed molecular diagnostics can be performed in the same FNAB sample.
It is not necessary to prepare part of or further FNAB material for RNA preservation.
It is not necessary to store part of the FNAB material or additional FNAB material for all patients undergoing FNA until completion of cytologic diagnosis to then only select those 20% stored samples with indeterminate or follicular proliferation cytology reports for further molecular analysis.
As it avoided to perform a second FNAB for molecular diagnostics, the method according to the invention is less of a burden for the patient.
The total diagnostic costs are lowered due to spared unnecessary parallel morphologic and molecular diagnostics (and lower total cost due to spared surgeries).

An air dried fine needle aspiration biopsy (FNAB) sample is a sample obtained by routine FNAB. FNAB is sometimes also referred to as fine needle aspiration cytology (FNAC). The sample is taken by inserting a thin, hollow needle (preferably ranging from 22 to 27 gauge—commonly, 25 gauge) into the tumor mass. As the name indicates, the biopsy technique uses aspiration to obtain cells and fluid from the tumor mass. The needle is gently moved back and forth through the lesion several (e.g. 3-6) times in different directions to obtain a representative sample of tissue. A representative sample preferably contains at least 6 groups comprising 6 to 8 thyroid cells each. The FNAB sample contains at least 20, preferably 30 thyroid cells and preferably a maximum of 200, more preferably up to 150 thyroid cells, even more preferably up to 100 thyroid cells, most often 20-50 or 50-100 thyroid cells.

FNAB are very safe, minor surgical procedures. Local anaesthesia is not routinely used for FNAB. If needed, a small amount (0.5 to 1.0 ml) of 1% lidocaine without epinephrine can be infiltrated locally to produce a skin wheal only, in order not to obscure the nodule.

Preferably the obtained FNAB material is directly expelled onto a glass microscope slide. A thin smear is prepared by using the second glass slide to gently press down and draw out the material to a feathered edge. The smear is air dried or fixed immediately preferably in 95% alcohol or other commercially available cytological spray fixative. The slide is stained with a common histological dye, like Papanicolaou stain or Mai Grünwald stain, and assigned a cytology code, e.g. C1—insufficient material to make a diagnosis; C2—benign; C3—indeterminate/follicular proliferation; C4—suspicious; C5—malignant.

Surprisingly the air dried or fixed FNAB sample, preferably a routine smear obtained and stained like described above, can be used in the method according to the invention for RNA extraction and analysis of gene rearrangements and/or miRNA.

Thus, preferably RNA isolation in the method according to the invention is performed after cytology analysis (thus after fixation and staining) of the air dried fine needle aspiration biopsy (FNAB) sample.

Alternatively to preparing a smear, the FNAB material obtained is processed by liquid based cytology. Unlike a traditional FNAB smear, where the cells are placed directly on a microscope slide, in liquid based cytology the FNAB sample is placed into a preservative fluid (also fixing the cells), preferably alcohol, e.g. methanol or ethanol-based. The vial is then sent to the laboratory for further processing e.g. by the T2000 automated processor according to the manufacturer's recommendations. Red blood cells in the FNAB sample are preferably deleted and the thyroid cells are collected, preferably by centrifugation, and applied to a carrier, preferably a slide. The FNAB sample can then be dried, stained and examined in the same manner as a traditional smear by a cytologist.

The same FNAB sample obtained and stained like described above by liquid based cytology can be used in the method according to the invention for RNA extraction and analysis of gene rearrangements, point mutations and/or miRNA. Alternatively, left over cells not used for making the first slide can be used in the method according to the invention for RNA extraction and analysis of gene rearrangements, miRNA and/or for the detection of point mutations.

Preferably rearrangements of RET/PTC and/or PAX8/PPARG (paired box 8/peroxisome proliferator-activated receptor gamma) are detected in the isolated RNA, preferably by reverse-transcribing the isolated mRNA into cDNA and subsequent PCR-analysis (preferably a Real-time PCR) with specific primers and/or oligonucleotide probes that allow the detection of the rearrangements. Primers and oligonucleotide probes are known from the state of the art.

Alternatively the rearrangements are detected by performing sequencing, in particular Pyrosequencing, on the cDNA obtained. Pyrosequencing is a method of DNA sequencing (determining the order of nucleotides in DNA) based on the “sequencing by synthesis” principle. It differs from Sanger sequencing, in that it relies on the detection of pyrophosphate release on nucleotide incorporation, rather than chain termination with dideoxynucleotides (see also Ronaghi M. 2001. Genome Research 11 (1): 3-11).

The RNA extracted contains in particular messenger RNA (mRNA) and miRNA. MicroRNAs (miRNAs) are short ribonucleic acid molecules (about 20-30 nucleotides long) that are post-transcriptional regulators that bind to complementary sequences on target messenger RNA transcripts (mRNAs), usually resulting in translational repression and gene silencing.

Preferably, miRNA is converted to cDNA by RT-PCR. The obtained cDNA is sequenced and from the obtained data differential expression of at least one miRNA sequence is analyzed.

Preferably the following miRNA (miR) are detected: miR-21, miR-181, miR-182, miR-187, miR-221, and/or miR-222.

In a preferred method according to the invention a classification of the tumor to benign or malignant is performed by assessment of the differential expression of at least one, preferably at least two, expression patterns of miRNA comprising the following nucleic acid sequences:

(SEQ ID No. 25)
TCGAGGAGCTCACAGTCTAGTAA,
(SEQ ID No. 26)
AATGTTTAGACGGGCTC,
(SEQ ID No. 27)
TACCCTGTAGAACCGAATTT,
(SEQ ID No. 28)
TCCTGTACTGAGCTGCCCCGAGA,
and/or
(SEQ ID No. 29)
AACATTCAACGCTGTCGGTGAA.

In this analysis, downregulation of miRNA including SEQ ID No. 25 is indicative for a benign tumor, downregulation of miRNA including SEQ ID No. 26 is indicative for a malignant tumor, downregulation of miRNA including SEQ ID No. 27 is indicative for a malignant tumor, downregulation of miRNA including SEQ ID No. 28 is indicative for a benign tumor and downregulation of miRNA including SEQ ID No. 29 is indicative for a benign tumor.

In a particularly preferred method according to the invention the classification of the tumor is performed by successively assessing the differential expression of miRNA according to SEQ ID No. 25 to 29 (starting with SED ID No. 25 and ending with SEQ ID No. 29).

Preferably, in addition to the assessment of the differential expression of at least one of the above miRNA isoforms according to SEQ ID No. 25-29, differential expression of at least one of the following miRNA isoforms comprising a nucleic acid sequence according to SEQ ID No. 30-39 is assessed in order to further improve the classification:

(SEQ ID No. 30)
TGGAAGACTAGTGATTTTGTTGT,
(SEQ ID No. 31)
TTCCCTTTGTCATCCTATGCCT,
(SEQ ID No. 32)
TGGAAGACTAGTGATTTTGTTGTT,
(SEQ ID No. 33)
AAACCGTTACCATTACTGAGTTT,
(SEQ ID No. 34)
TGTAAACATCCTCGACTGGA,
(SEQ ID No. 35)
TGAGAACTGAATTCCATAGGCTG,
(SEQ ID No. 36)
ACCGGGTGCTGTAGGCTT,
(SEQ ID No. 37)
GAGAAAGCTCACAAGAACTG,
(SEQ ID No. 38)
CCTGTCTGAGCGTCGCT,
and/or
(SEQ ID No. 39)
TGAGAACTGAATTCCATAGGCTGT.

Here, miRNA isoforms comprising a nucleic acid sequence according to SEQ ID No. 30-34 are upregulated in benign tumor samples and miRNA isoforms comprising a nucleic acid sequence according to SEQ ID No. 35-39 are upregulated in malignant tumor samples.

Preferably, in addition to the analysis of differential expression of at least one of the above miRNA isoforms according to SEQ ID No. 25-29 (and preferably also of at least one of the above miRNA isoforms according to SEQ ID No. 30-39) at least one miRNA comprising one of the following miRNA seed sequences (the 5′ bases 1-8 of the miRNA) is quantified in order to further improve the classification:

(SEQ ID No. 40)
CCTGTCTG,
(SEQ ID No. 41)
GAGAAAGC,
(SEQ ID No. 42)
ATGTTTAG,
(SEQ ID No. 43)
TGGAAGAC,
(SEQ ID No. 44)
TTCCCTTT,
(SEQ ID No. 45)
GTCCAGTT,
(SEQ ID No. 46)
AACCCGTA,
(SEQ ID No. 47)
AAACCGTT,
and/or
(SEQ ID No. 48)
TCCTGTAC.

Here, miRNA comprising one of the miRNA seed sequences according to SEQ ID No. 40-42 are upregulated in malignant tumor samples and miRNA comprising one of the miRNA seed sequences according to SEQ ID No. 43-48 are upregulated in benign tumor samples.

The invention also includes the use of miRNA comprising a nucleic acid sequence according to one SEQ ID No. 25-48, preferably one of SEQ ID No. 25-29, as marker for distinguishing malignant from benign tumor samples of the thyroid.

The analysis of miRNA is preferably performed quantitatively. Methods for specific RNA detection are known. The detection is preferably done with primers and/or oligonucleotide probes hybridising to the RNA or after reverse transcription to the corresponding cDNA. The oligonucleotide probes might be labelled or part of a microarray (e.g. a miRNACHIP). In the latter case the RNA isolated is preferably labelled (e.g. biotinylated) and incubated with the microarray.

Preferably, in the method according to the invention RNA, in particular mRNA and miRNA, and genomic DNA are extracted simultaneously. It is a particular advantage of this embodiment that RNA (mRNA and/or miRNA) and genomic DNA is extracted from one sample, as it reduces the numbers of biopsies.

The extracted DNA is preferably used to detect point mutations, in particular in DNA encoding BRAF, N-, K-, and/or HRAS. Commercial assays for the detection of BRAF, KRAS and NRAS mutations can be used.

Preferably mutations selected from the following list are detected:
BRAF: V600E, K601E (protein), as well as T1796A and T1799A (DNA),
NRAS: any mutation in codon 61,
KRAS: any mutation in codon 12 and codon 13,
HRAS: any mutation in codon 61.

After PCR amplification point mutations are preferably detected with high-resolution-melting (HRM) analysis or Pyrosequencing. HRM is more sensitive than FRET based analysis and Sanger sequencing. The advantages of pyrosequencing are a high sensitivity and more objective results regarding the mutational status since both qualitative and quantitative information are given.

The presence of a gene-rearrangement and/or the differential expression of miRNA is indicative for a malignant tumor.

In particular, the presence of RET/PTC and/or PAX8/PPARG rearrangements and/or mutations in genes encoding BRAF, N-, K-, and/or HRAS and/or differential expression of miRNA in the sample classifies the tumor as malignant. Moreover, miRNA quantification and the miRNA classifier identify benign samples. Differential expression means that the expression is higher or lower than in healthy tissue, in particular of the thyroid.

The detection of any of the point mutations or rearrangements or a differential expression of miRNA or the miRNA classifier gives an indication for total thyroidectomy including central lymph node compartment dissection whereas their absence and especially a benign miRNA expression or benign miRNA classifier result would argue for follow up.

Another aspect of the invention is the use of a kit for carrying out the method of the invention.

This kit preferably contains at least one of the following components:
i. Buffer for nucleic acid extraction,
ii. Primer (e.g. oligo-dT, random hexamer, target specific reverse transcription primers), buffers and RNA-dependent-DNA-polymerase for Reverse transcription (RT),
iii. Primers (e.g., PCR primers and/or sequencing primers) and optionally oligonucleotide probes (preferably single or dual Fluorescence-labeled), Polymerase (preferably taq polymerase or another thermostable DNA-dependent DNA polymerase) and buffers for PCR on cDNA to detect gene rearrangements, in particular RET/PTC and/or PAX8/PPARG gene rearrangements preferably be real-time PCR or pyrosequencing,
   and optionally:
iv. Primers (e.g., PCR primers and/or sequencing primers and optionally oligonucleotide probes), Polymerase (preferably taq polymerase or another thermostable DNA-dependent DNA polymerase) and buffers for PCR on genomic DNA to detect point mutations, in particular in genes encoding BRAF, N-, K-, and/or HRAS and/or
v. oligonucleotide probes (preferably single or dual Fluorescence-labeled) for the detection of miRNA, and/or
vi. Primers (e.g., PCR primers and/or sequencing primers and optionally oligonucleotide probes), Polymerase (preferably taq polymerase or another thermostable DNA-dependent DNA polymerase) and buffers for PCR on cDNA to detect and quantify miRNA.

The primers for PCR and the oligonucleotide probes, in particular used for real time PCR or detection of the miRNA, are oligonucleotides (preferably with a length of 15 to 25 nucleotides) that are complementary to the target sequence (nucleic acid sequence to be detected) and specifically hybridize thereto by complementary base paring. The oligonucleotide probes are preferably labeled, e.g. with dyes (in particular fluorescent dyes), haptens (such as biotin or digoxigenin) or radioactively.

Alternatively the oligonucleotide probes are part of a microarray, e.g. a miRNACHIP. In this case the RNA isolated is preferably labelled (e.g. biotinylated) and incubated with the microarray.

Thus, in this case the kit preferably contains additionally or alternatively at least one of the following components:
i. Buffer for nucleic acid extraction,
ii. Reagents for labeling RNA and/or DNA, in particular fluorescent dyes,
iii. Hybridization buffers,
iv. Washing buffers.

The invention is further illustrated by the following figures and examples without being limited to these.

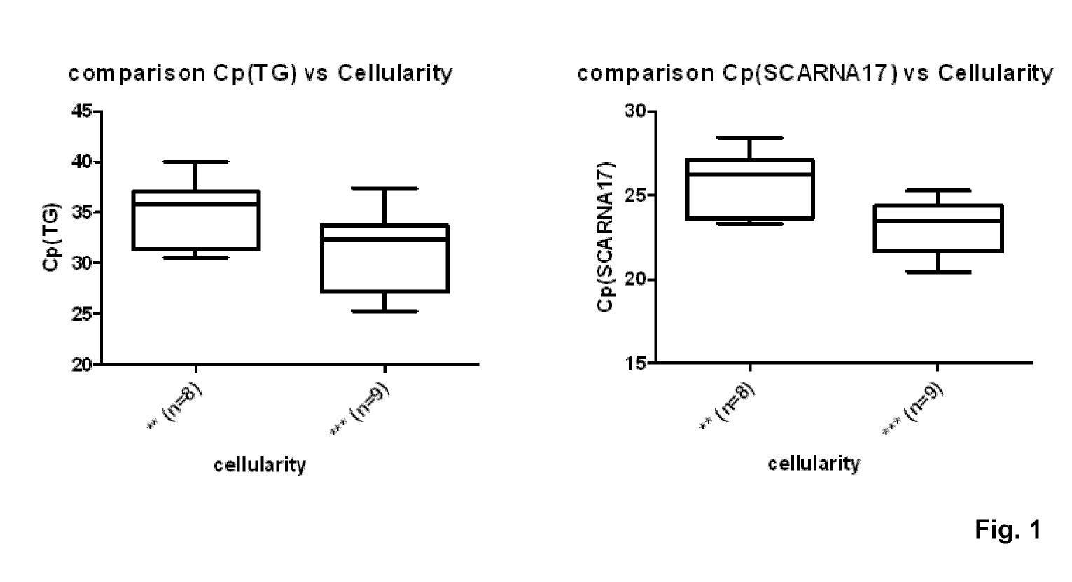

FIG. 1 shows the results of Thyroglobulin (TG) mRNA and SCARNA17 expression analysis in comparison to the cellularity of the samples. (++=20-50 thyroid epithelial cells, +++>50 thyroid epithelial cells).

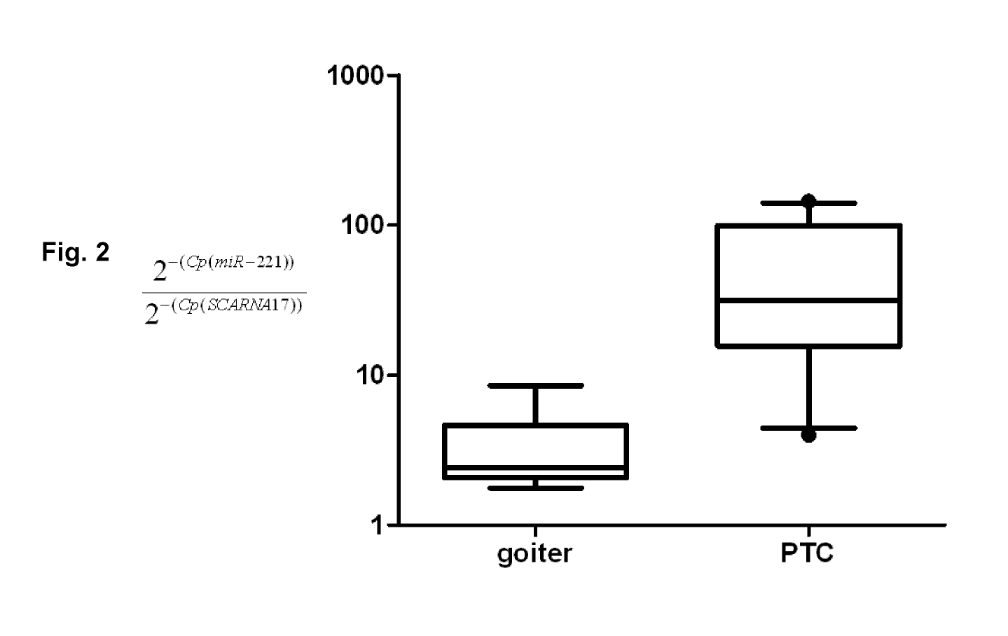

FIG. 2 shows the results of miR-221 expression analysis in PTC versus goiter. miR-221 (normalized to SCARNA17) shows a significantly increased expression in PTC versus goiter (p<0.001).

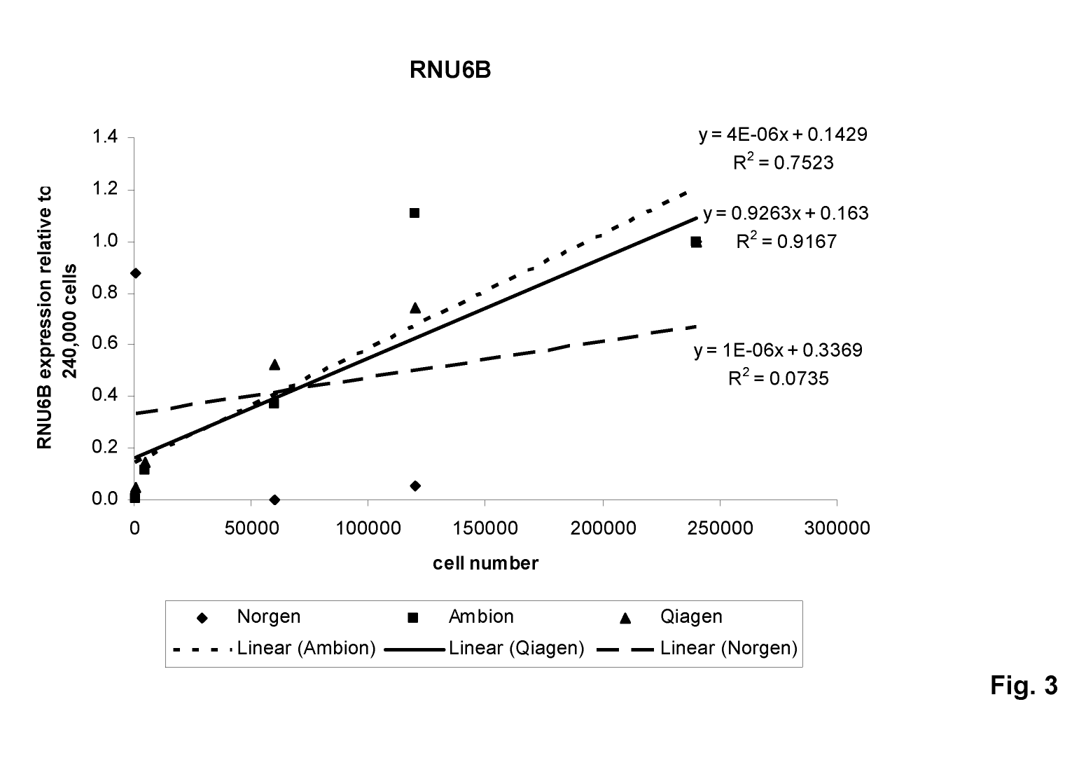

FIG. 3 shows the results of quantification of miRNA housekeeping RNA (RNU6B).

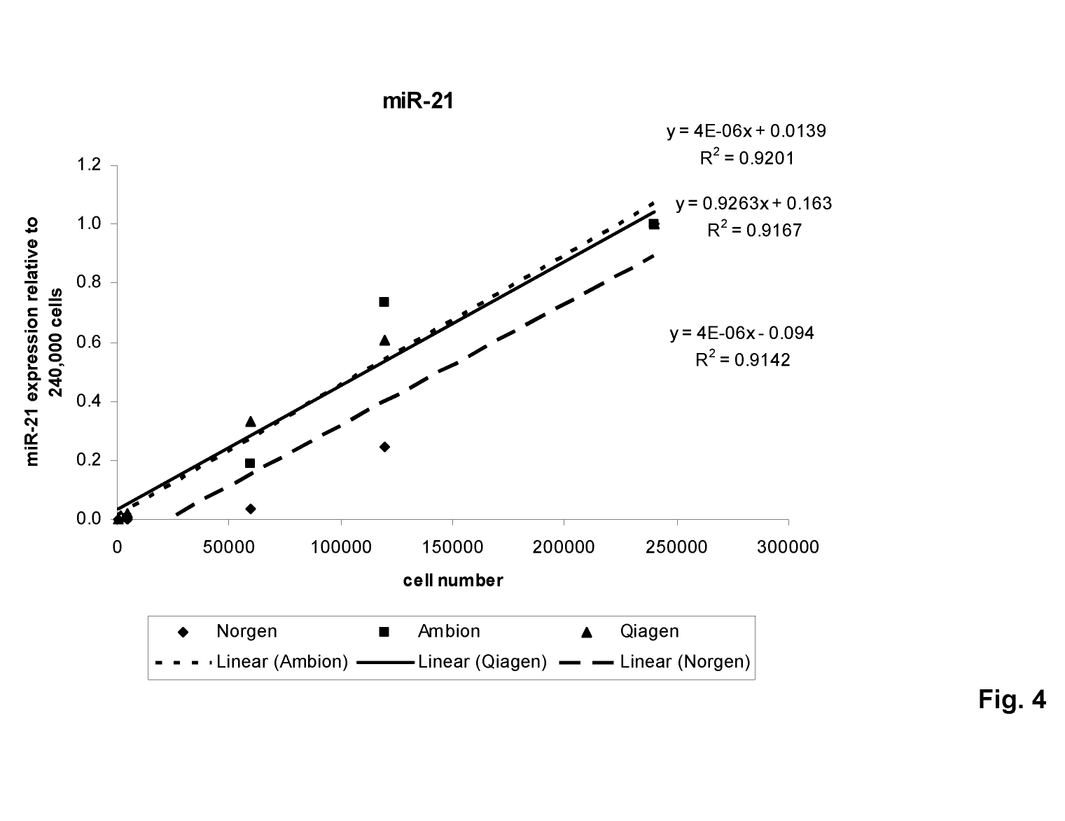

FIG. 4 shows the results of quantification of miRNA miR-21.

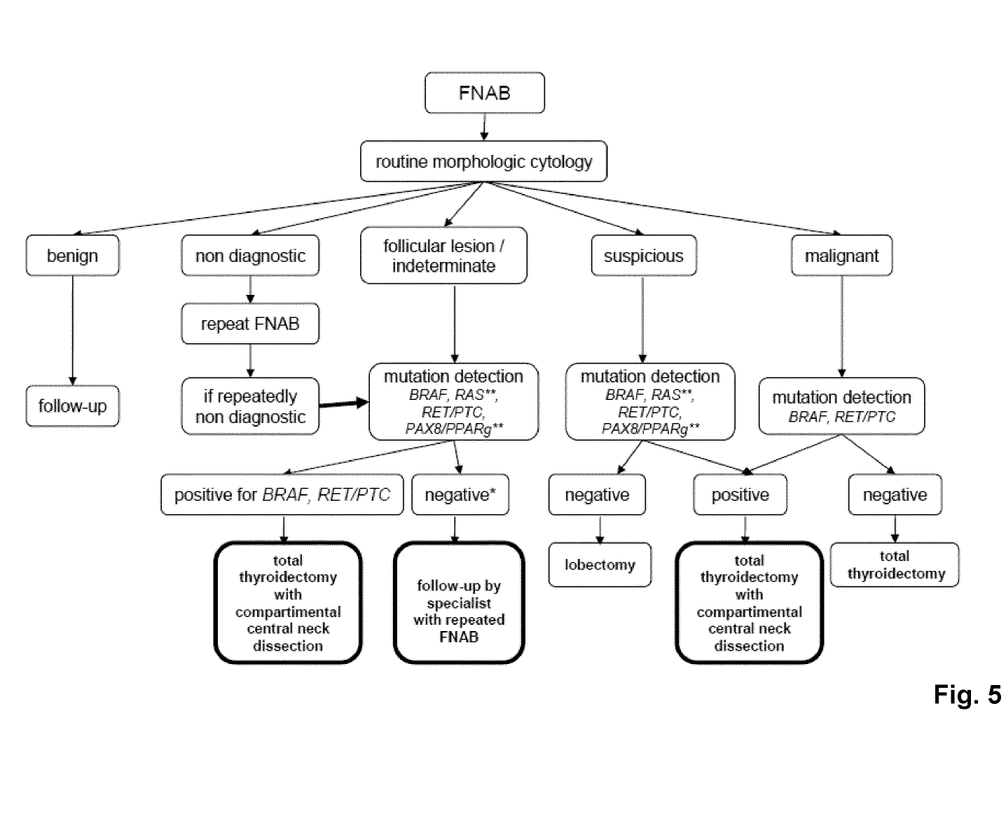

FIG. 5 shows the additional clinical consequences of the molecular diagnostic provided by the invention in bold.

* optional further diagnosis by miRNA markers,
** if positive for RAS mutation or PAX8/PPARg rearrangement lobectomy in general justified.

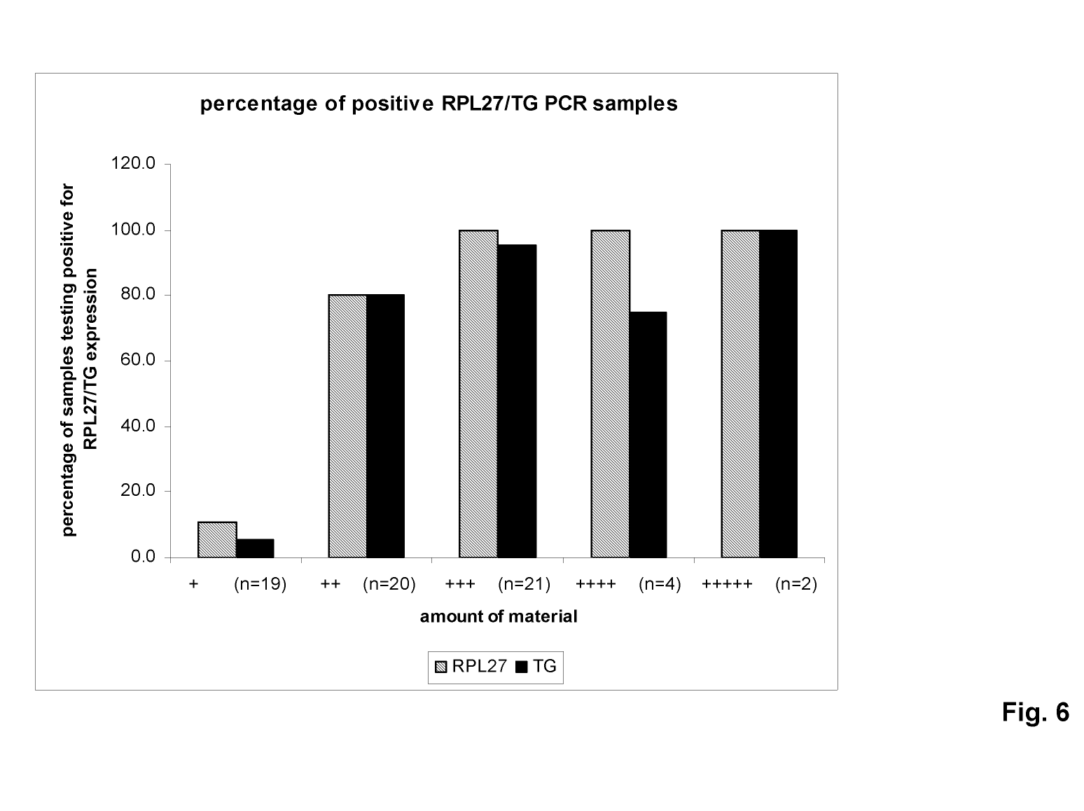

FIG. 6 shows the results of RPL27 and TG mRNA expression analysis in samples process.

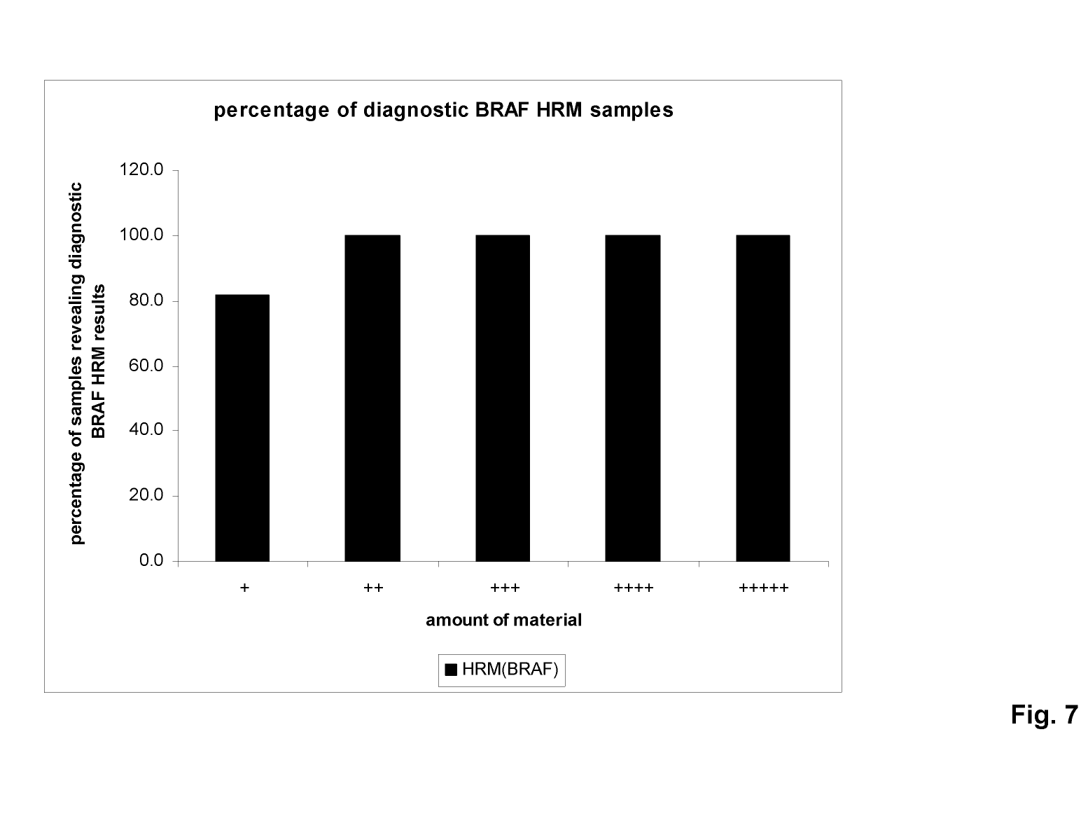

FIG. 7 shows the results of a screening for BRAF mutations by HRM.

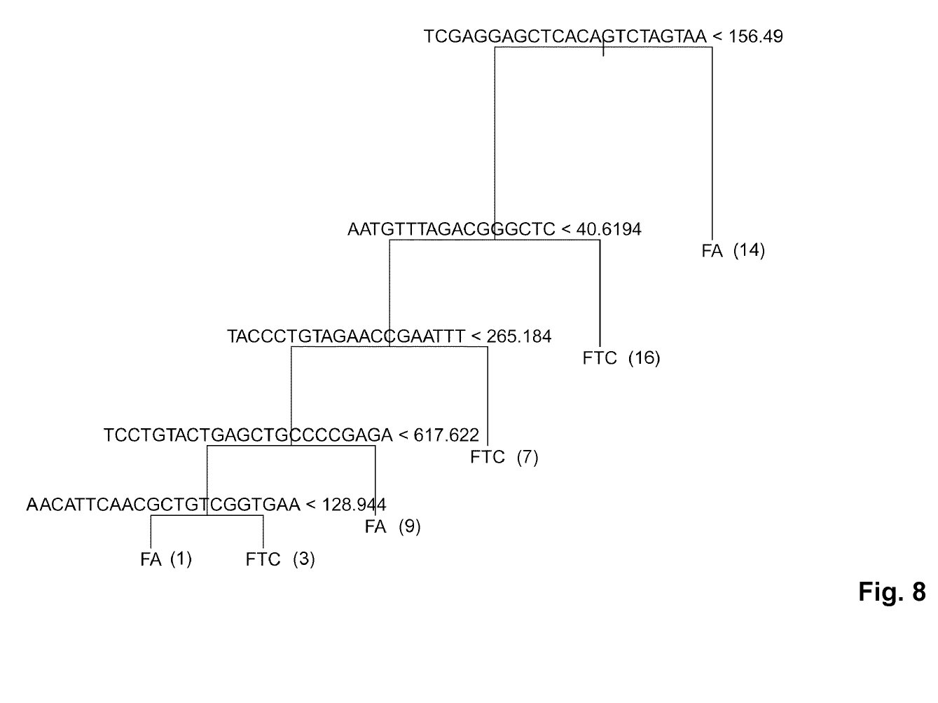

FIG. 8 shows the decision tree for the classification of a set of 25 FTA (benign) and 25 FTC (malignant) using the miRNA classifier. On top the miRNA sequence is shown and the thresholds for miRNA expression are given. Here, expression of the respective miRNA sequence with a RPM (reads per million) below the threshold is indicative for the malignant (FTC) or benign (FA) tumor as indicated. The number of FTA and FTC samples classified per branch is given in brackets.

### 1. SETTING-UP DNA, MRNA, AND MIRNA EXTRACTION METHODS FROM ROUTINE FNAB

To carry out the method according to the invention, methods for extracting RNA, particularly miRNA, and DNA from air dried or fixed FNAB samples were optimized to assure a quantitative miRNA extraction.

There are currently two commercial kits marketed for the extraction of DNA, mRNA, and miRNAs, Ambion RecoverAll Total Nucleic Acid Isolation Kit, Norgen All-in-One Purification Kit). At first, the extraction capabilities of these two kits and also the Qiagen miRNeasy Mini Kit (which is only marketed for mRNA and miRNA extraction), especially the quantitative recovery of miRNAs, were tested with dilution series of GripTite™ 293 MSR cells (Invitrogen Corp., Carlsbad, Calif.) ranging from 480 to 240,000 cells. RNA was extracted from the cells according to the respective manufacturer's instructions. Subsequent to extraction, the RNA was reverse transcribed using the QIAGEN miScript Reverse Transcription Kit according to the manufacturer's instructions. Afterwards a miRNA housekeeping RNA (RNU6B), and a further miRNA (miR-21) were quantified on a Roche Lightcycler 480 using the QIAGEN miScript Primer Assays (RNU6B: catalog number: MS00029204, miR-21: catalog number: MS00009079) according to the manufacturer's instructions. The results of these quantifications are shown in FIGS. 3 and 4. While there are no significant differences for the correlation coefficient of miR-21 expression and the cell number (FIG. 4) for the three different extraction methods used there are strong differences for the correlation coefficients for RNU6B expression. The kit showing the best correlation for RNU6B expression in relation to the cell number is the QIAGEN miRNeasy Mini Kit (R²=0.92) which was also shown to allow a quantitative extraction of miRNAs. Therefore, this kit was modified in a way that allows also DNA extraction as outlined above, and was subsequently used for all extractions.

Subsequently, 20 FNAB slides (10 PTCs, 10 goiters) were used to further evaluate the best performing extraction kit (QIAGEN miRNeasy Mini Kit) for its efficiency regarding DNA and m/miRNA recovery. In detail, the co-extraction of DNA and RNA from FNAB samples was done as follows. First, the FNAB slides were incubated in Xylol for 4-5 d to remove the cover slips. Afterwards, the slides were air dried. Subsequent, 700 μl Qiazol™ (Phenol and Guanidiniumthiocyanat containing lysis reagent by Qiagen, Hilden, Germany) were added to the slide according to the miRNeasy kit (Qiagen, Hilden, Germany) protocol and the cells on the slide were lysed within the Qiazol™ using a scalpel. The lysed cells were transferred to a new tube, homogenized by pipetting up and down/vortexing. 240 μl Chloroform were added, mixed for 15 sec and subsequently incubated at room temperature (RT) for 3 min. Then, centrifuge at full speed at 4° C. for 15 min. The upper phase was transferred to a new tube and extraction was continued according to the miRNA kit (Qiagen, Hilden, Germany) protocol. The mi/mRNA was eluted in 40 μl ad.

Moreover, to also extract DNA from the same sample from the first tube rests of the upper phase were removed and 300 μl 96% Ethanol was added. The tube was gently mixed and afterwards centrifuged at 8,000 g for 3 min. The supernatant was removed and the pellet was incubated with sodium citrate solution for 30 min. Afterwards, the tube was centrifuged at 8,000 g for 3 min and again incubated with sodium citrate solution for 30 min. After a centrifugation at 8,000 g for further 3 min at RT the pellet was washed with 70% Ethanol. Subsequent to a further centrifugation the pellet was dried at RT for 15 min and then resuspended in 50 μl TE buffer (10 mmol/l Tris-Cl, pH 7.5. 1 mmol/l EDTA). After freezing the DNA for 24 hours it was thawed, vortexed and centrifuged at max. speed for 1 min. The supernatant containing the DNA was transferred to a new tube.

### 2. QUANTIFICATION OF mRNA/miRNA

To check the m/miRNA quality and recovery, Thyroglobulin (TG) mRNA as well as the small housekeeping genes were quantified by real time PCR on a Roche LightCycler 480 and the results were compared with the number of thyroid cells graded by the pathologist. TG mRNA could be amplified in 19 out of the 20 test FNAB slides and the small housekeeping RNAs could be amplified in all 20 test FNAB samples. Furthermore a correlation of the expression of TG mRNA and the small housekeeping RNAs and the cellularity (++=20-50 thyroid epithelial cells, +++>50 thyroid epithelial cells) of the test samples could be shown (FIG. 1).

The quantification of TG mRNA by real time PCR was performed using a LightCycler 480 (Roche, Mannheim, Germany). Oligonucleotide primers were designed to be intron spanning and were purchased from MWG Biotech AG (Ebersberg, Germany). Sequences were obtained from the GenBank database. The nucleotide sequences of the two primers are:

TG-Forward:
(SEQ ID No. 1)
5′-CCTGCTGGCTCCACCTTGTTT-3′
and
TG-Reverse:
(SEQ ID No. 2)
5′-CCTTGTTCTGAGCCTCCCATCGTT-3′.

PCRs were performed using the LightCycler DNA Master SYBR Green I Kit (Roche, Mannheim, Germany) according to the manufacturer's instructions. The TG-PCR was processed through 45 cycles including 5 sec of denaturation at 95° C., a 7 sec annealing phase at 62° C. and an elongation phase at 72° C. for 7 sec. A 20 μl reaction consisted of 2 μLightCycler FastStart DNA Master SYBR Green I (containing Taq DNA Polymerase, reaction buffer, dNTP mix (with dUTP instead of dTTP) and 10 mmol/l MgCl₂), additional 1.6 μl MgCl₂, 0.5 μM of each primer, and 2 μl of template.

Moreover, miR-221 which is known to be up-regulated in PTC, was quantified in the 20 FNAB slides to study
i) whether miRNA can be quantitatively extracted from routine FNAB slides,
ii) whether miRNA can be quantified in routine FNAB slides, and
iii) whether a discrimination between benign and malignant samples can be done based on the quantification of miRNA in routine FNAB slides.

MiR-221 has the following Sequence (Entrez Gene ID 407006):

(SEQ ID No. 3)
5′-AGCUACAUUGUCUGCUGGGUUUC-3′.

MiR-221 showed a significantly increased expression (p<0.001) in the PTC-FNAB samples compared to the goiter-FNAB samples confirming the data known from the literature and suggesting a successful quantification of miRNAs in RNA samples from FNABs (FIG. 2). These data show that miRNA could be quantitatively extracted from routine FNAB slides, that miRNA could be quantified in routine FNAB slides and that it was possible to discriminate between benign and malignant samples based on the quantification of miRNA in routine FNAB slides.

DNA was extracted from the left-over of the m/miRNA extraction and DNA quality was checked by amplifying a BRAF fragment, which was possible in all 20 samples. The quantification of BRAF genomic DNA (gDNA) by real time PCR was performed using a LightCycler 480 (Roche, Mannheim, Germany). Primer sequences for the amplification of BRAF were BRAF-F: 5′-TCATAATGCTTGCTCTGATAGGA-3′ (SEQ ID No. 4) and BRAF-R: 5′-GGCCAAAAATTTAATCAGTGGA-3′ (SEQ ID No. 5) according to Nikiforov et al. 2009 (J Clin Endocrinol Metab 94(6):2092-2098). PCRs were run using the LightCycler DNA Master SYBR Green I Kit (Roche, Mannheim, Germany) according to the manufacturer's instructions. The BRAF-PCR was processed through 45 cycles including 5 sec of denaturation at 95° C., a 7 sec annealing phase at 55° C. and an elongation phase at 72° C. for 9 sec. A 20 μl reaction consisted of 2 μl LightCycler FastStart DNA Master SYBR Green I (containing Taq DNA Polymerase, reaction buffer, dNTP mix (with dUTP instead of dTTP) and 10 mmol/l MgCl₂), additional 1.6 MgCl₂, 0.5 μmol/l of each primer, and 2 μl of template.

### 3. DETECTION OF RET/PTC AND PAX8/PPARG IN ROUTINE FNAB SLIDES

100 routine air dried FNAB slides from patients who underwent surgery for thyroid nodules at the Odense University Hospital were retrospectively included into this study. In addition, 100 patient matching formalin-fixed paraffin embedded (FFPE) slices were analyzed. All FNAB samples were graded according to the ATA 2006 guidelines by two pathologists. In detail, cytologic evaluation of the FNAB slides revealed 28 malignant, 55 indeterminate, 16 non-neoplastic, and 1 non-diagnostic samples. Histologic evaluation of the corresponding FFPE samples revealed 45 follicular adenoma (FA), 10 oncocytic FA, 4 follicular thyroid carcinoma (FTC), 6 oncocytic FTC, 22 papillary thyroid carcinoma (PTC), 3 follicular variants of PTC (fvPTC), and 10 goiters.

RNA was extracted and reverse transcribed from routine air dried FNAB smears as described above. cDNA was synthesized using the miScript Reverse Transcription Kit (Qiagen, Hilden, Germany) according to the manufacturers suggestions. In brief, 7.5 μl template RNA were added to a master mix consisting of 2 μl 5×miScript RT buffer and 0.5 μl miScript Reverse Transcriptase Mix and incubated for 60 min at 37° C. Subsequently, the miScript Reverse Transcriptase Mix was inactivated for 5 min at 95° C. To check the RNA quality, an intron-spanning 122 bp fragment of PAX8 mRNA (exon 5-6) was analyzed by real time PCR with subsequent fluorescence melting curve analysis on a Roche LightCycler 480 using FastStart SYBR Green Master chemistry (Roche, Mannheim, Germany). PAX8/PPARG, RET/PTC1 and RET/PTC3 rearrangements were detected by real time PCR using previously described primers and probes flanking the fusion points (Algeciras-Schimnich A et al. 2010 Clin Chem 56(3):391-398 and Nikiforov Y E et al. 2009. J Clin Endocrinol Metab 94(6):2092-2098) (Table 1) and the LightCycler FastStart DNA MasterPlus HybProbe chemistry (Roche, Mannheim, Germany). These primer/probe combinations allow to detect all four described translocations between PAX8 and PPARG: PAX8(exon 1-8)/PPARG (PAX8-E8-F/PPARG-E1-R, 85 bp), PAX8(exon 1-9)/PPARG (PAX8-E9-F/PPARG-E1-R, 115 bp), PAX8(exon 1-10)/PPARG (PAX8-E10-F/PPARG-E1-R, 95 bp), and PAX8(exon 1-8,10)/PPARG (PAX8-E10-F/PPARG-E1-R, 188 bp). Moreover, RET/PTC1 (RET/PTC1-F/RET/PTC1-R, 136 bp) and RET/PTC3 (RET/PTC3-F/RET/PTC3-R, 106 bp) could be detected. PCRs were processed through an initial denaturation at 95° C. for 5 min followed by 50 cycles of a 3-step PCR, including 10 sec of denaturation at 95° C., a 10 sec annealing phase at 62° C. (PAX8/PPARG) or 64° C. (RET/PTC) and an elongation phase at 72° C. for 7 seconds. cDNA from patient specimens known to carry PAX8/PPARG or RET/PTC rearrangements were used as positive controls in each analysis. Positive tested samples were analyzed by capillary gel electrophoresis using BigDye Terminator Kit on an ABI 3100 Genetic Analyzer (Applied Biosystems).

TABLE 1
Primers for the detection of RET/PTC and PAX8/PPARG rearrangements
and the PCR control PAX8

| | | | SEQ ID |
|---|---|---|---|
| Primer | GenBank ID | sequence | No. |
| PAX8-E5-F | NM_003466 | TCAACCTCCCTATGGACAGC | 6 |
| PAX8-E6-R | NM_003466 | GGAGTAGGTGGAGCCCAGG | 7 |
| PAX8-E8-F | NM_003466 | CCTCTCGACTCACCAGACCT | 8 |
| PAX8-E9-F | NM_003466 | GCCCTTCAATGCCTTTCCCCATG | 9 |
| PAX8-E10-F | NM_003466 | AGCGGACAGGGCAGCTATGC | 10 |
| PPARG-E1-R | NM_138712 | CCAAAGTTGGTGGGCCAGAAT | 11 |
| PPARG-Probe | NM_138712 | FAM-CATGGTTGACACAGAGAT-BHQ1 | 12 |
| RET/PTC1-F | NM_005436 | GGAGACCTACAAACTGAAGTGCAA | 13 |
| RET/PTC1-R | NM_020975 | CCCTTCTCCTAGAGTTTTTCCAAGA | 14 |
| RET/PTC1-Probe | NM_005436 | FAM-AACCGCGACCTGCGCAAAGC-BHQ1 | 15 |
| RET/PTC3-F | NM_005437 | CCAGTGGTTATCAAGCTCCTTACA | 16 |
| RET/PTC3-R | NM_020975 | GGGAATTCCCACTTTGGATCCTC | 17 |
| RET/PTC3-Probe | NM_005437 | FAM-ACCCAGCACCGACCCCCAGG-BHQ1 | 18 |

FAM: Fluorescein; BHQ1: Black Hole Quencher 1.

Three FNAB samples were tested positive for RET/PTC1 and confirmed by Sanger sequencing. One of these samples was a PTC whose FFPE sample was also RET/PTC1 positive. Two FNAB positive samples are histologically FA and the rearrangement could not be detected in the FFPE samples. One PTC sample was tested positive for RET/PTC3 both in the FNAB and the FFPE sample. Moreover, one further RET/PTC3 rearrangement was detected in a FFPE sample of a FA but the corresponding FNAB sample was mutation negative.

PAX8/PPARG was detected by RT-PCR in 7 of 76 FFPE samples (9%) and in 6 of 76 FNAB smear samples. In 4 samples it was possible to match FFPE to FNAB (3 FA and 1 FTC). 3 rearrangement positive FFPE cases could not be detected in FNAB, and 2 positive smear samples could not be identified in the corresponding FFPE. PAX8/PPARG was present in 2 of 6 (33%) FTC; 5 of 29 follicular adenomas (17%) and also in 1 Hurthle cell adenoma (n=8 in total). No rearrangement was detected in Hurthle cell carcinomas (n=1), goiter (n=7) or PTC (n=25). The most frequent fusion variant was PAX8 exons 1-8 juxtaposed to PPARg exon 1 (55%), followed by PAX8 exons 1-9 juxtaposed to PPARg exon 1. The least frequent variant was PAX8 exons 1-10 juxtaposed to PPARg exon 1 (16.7%).

These results demonstrate the feasibility of extracting RNA from routine air dried FNA smears to detect PAX8/PPARg rearrangements with RT-PCR. The introduction of molecular analyses of routine air dried FNA smears in every day practice, comprising also other mutations, could provide substantial improvements for the diagnosis of thyroid cancer and thereby potentially also reduce the rate of diagnostic surgery.

### 4. DETECTION OF BRAF, H-, K-, AND NRAS MUTATIONS IN ROUTINE FNAB AND FFPE SLIDES USING HYBRIDIZATION PROBES

DNA extracted from the FNAB and FFPE samples was screened for the point mutations BRAF V600E and K601E, and for point mutations in KRAS codons 12/13, and NRAS codon 61 by real time PCR using hybridization probes and fluorescence melting curve analysis on a Lightcycler 480 according to Nikiforov Y E et al. 2009 (cited above). The PCRs for the detection of these point mutations were applicable to our DNA samples, which are (due to the extraction from routine FNAB and FFPE samples) of lower quality than the DNAs extracted from fresh FNAB material.

50 FNAB and FFPE samples have been screened for the BRAF V600E mutation using hybridization probes and fluorescence melting curve analysis:

TABLE 2

| BRAF | FNAB | FFPE |
|---|---|---|
| positive in mutation screening | 5 | 10 |
| wildtyp in mutation screening | 15 | 32 |
| questionable | 10 | 4 |
| non-diagnostic (no PCR-product/low efficiency PCR) | 20 | 4 |

In summary, a BRAF mutation could be detected in 29% of the PTC-FNAB samples and in 47% of the PTC-FFPE samples. However, the BRAF detection in the FNAB samples can be further improved by High Resolution Melting (HRM) analysis to reduce the number of questionable and non-diagnostic FNAB samples:

The same 50 FNAB and FFPE samples screened by hybridization probes according to Nikiforov et al. were screened by HRM analysis again. PCRs were processed through an initial denaturation at 95° C. for 10 min followed by 50 cycles of a 3-step PCR, including 3 sec of denaturation at 95° C., a 10 sec annealing phase at 58° C. and an elongation phase at 72° C. for 10 seconds on a LightCycler 480. Subsequently a high resolution melting curve was assessed. The following primers were used:

BRAF-F:
(SEQ ID No. 19)
5′-GGTGATTTTGGTCTAGCTACAG-3′
and
BRAF-R:
(SEQ ID No. 20)
5′-GGCCAAAAATTTAATCAGTGGA-3′.

The results of the HRM assay are as follows:

TABLE 3

| BRAF | FNAB | FFPE |
|---|---|---|
| positive in mutation screening | 9 | 12 |
| wildtyp in mutation screening | 40 | 35 |
| questionable | 0 | 3 |
| non-diagnostic (no PCR-product/low efficiency PCR) | 1 | 0 |

The comparison of the two methods shows that HRM analysis detects more BRAF mutations (FNAB: 9 vs. 5, FFPE: 12 vs. 10) and results in less questionable/non-diagnostic results (FNAP: 0/1 vs. 10/20, FFPE: 3/0 vs. 4/4). In summary, a BRAF mutation could be detected in 29% of the PTC-FNAB samples by using hybridization probes and 47% of the PCT-FNAB samples by HRM. Furthermore, a BRAF mutation could be detected in 47% of the PTC-FFPE samples by using hybridization probes and 70% of the PCT-FFPE samples by HRM. The occurrence of mutations was verified by Sanger sequencing. These results clearly indicate that HRM is superior to the use of hybridization probes in detecting BRAF point mutations.

So far NRAS-screening revealed that 3 FA of all screened FNAB-samples carried a NRAS-mutation, whereas 15 (22.7%) of the FFPE-samples were tested positive for NRAS mutation.

TABLE 4

| NRAS | FNAB | FFPE |
|---|---|---|
| positive in mutation screening | 3 | 15 |
| wildtyp in mutation screening | 53 | 47 |
| non-diagnostic (no PCR-product/low efficiency PCR) | 10 | 4 |

NRAS mutations can also be detected in an High Resolution Melting (HRM) assay as described for BRAF and KRAS.

Up to now 70 samples were screened for point mutations in KRAS codons 12/13 using hybridization probes PCR. No KRAS-mutation was thus far detected in FNAB- and FFPE samples and approximately three-third of all screened FNAB- and FFPE-samples were WT. Compared with the 11 non-diagnostic FNAB-samples (15.7%) that showed no or a weak amplification in the PCR, there where 10 FFPE-samples (14.2%) with the same problems.

TABLE 5

| KRAS | FNAB | FFPE |
|---|---|---|
| positive in mutation screening | 0 | 0 |
| wildtyp in mutation screening | 53 | 59 |
| questionable | 6 | 1 |
| non-diagnostic (no PCR-product/low efficiency PCR) | 11 | 10 |

A High Resolution Melting (HRM) assay was established also to detect KRAS mutations and the results of both methods were compared.

The same 70 FNAB and FFPE samples screened by hybridization probes according to Nikiforov et al. 2009 (cited above) were screened by HRM analysis again. PCRs were processed through an initial denaturation at 95° C. for 10 min followed by 50 cycles of a 3-step PCR, including 3 sec of denaturation at 95° C., a 12 sec annealing phase at 58° C. and an elongation phase at 72° C. for 10 seconds on a Lightcycler 480. Subsequently a high resolution melting curve was assessed. The following primers were used:

KRAS-F:
(SEQ ID No. 21)
5′-AGGCCTGCTGAAAATGACTG-3′
and
KRAS-R:
(SEQ ID No. 22)
5′-GCTGTATCGTCAAGGCACTCT-3′.

The results of the HRM assay are as follows:

TABLE 6

| KRAS | FNAB | FFPE |
|---|---|---|
| positive in mutation screening | 3 | 1 |
| wildtyp in mutation screening | 55 | 48 |
| questionable | 0 | 13 |
| non-diagnostic (no PCR-product/low efficiency PCR) | 12 | 8 |

In summary, the experiments show that the PCRs using hybridization probes for mutation detection could be applied also to DNAs of lower quality. High resolution melting (HRM) analysis for detection of the point mutations is preferred.

### 5. LIQUID CYTOLOGY—QUANTIFICATION OF TG AND RPL27 mRNA IN RELATION TO THE AMOUNT OF LIQUID BASED FNAB MATERIAL

For liquid based cytology the material obtained by FNAB is expelled into a cell preservation solution (methanol based Cytolyt™ solution, Cytyc Corp. Marlborough, Mass., USA). Thereafter, the cells are spun and the pellet is transferred into preservCyt™ (Cytyc Corp) for further processing in the T2000 automated processor (Cytyc Corp) according to the manufacturer's recommendations. A thin evenly dispersed monolayer of cells was dispersed from the filter onto the slide in a cycle of 20 mm in diameter (ThinPrep slides). ThinPrep slides were stained and histology was performed.

The cells not used for making the slide are stored in the cell preservation solution e.g. preserveCyt™ and subsequently used for parallel RNA (in particular mRNA and miRNA) and DNA extraction. Alternatively, RNA and DNA extraction is performed with the cells removed from the slide after staining and histology.

For RNA ad DNA extraction the samples were transferred to Falcon tubes and pelleted by centrifugation. Afterwards, the supernatant was removed and 700 μl Qiazol (Qiagen, Hilden, Germany) were added to the pellet according to the miRNeasy kit protocol and the cells were lysed within the Qiazol. The lysed cells were transferred to a new tube, homogenized by pipetting up and down/vortexing. 240 μl Chloroform were added, mixed for 15 sec and subsequently incubated at room temperature for 3 min. Then, the samples were centrifuged at full speed at 4° C. for 15 min. The upper phase was transferred to a new tube and extraction was continued according to the miRNA kit protocol. The mi/mRNA was eluted in 40 μl dad.

From the first tube rests of the upper phase were removed and 300 μl 96% Ethanol added. The tube was gently mixed and afterwards centrifuged at 8,000 g for 3 min. The supernatant was removed and the pellet was incubated with sodium citrate solution for 30 min. Afterwards, the tube was centrifuged at 8,000 g for 3 min and again incubated with sodium citrate solution for 30 min. After a centrifugation at 8,000 g for further 3 min at room temperature the pellet was washed with 70% Ethanol. Subsequent to a further centrifugation the pellet was dried at room temperature for 15 min and then resuspended in 50 μl TE buffer. After freezing the DNA for 24 hours it was thawed, vortexed and centrifuged at max. speed for 1 min. The supernatant containing the DNA was transferred to a new tube.

The amount of liquid based FNAB material supplied was documented as “+—minimal amount of material visible” to “+++++—very high amount of material”. DNA and RNA was extracted from all samples. To check the extracted RNA and DNA, the RNA was reverse transcribed (as described above) and TG (as described above) and the housekeeping gene RPL27 were quantified by real time PCR. Moreover, using the extracted DNA BRAF was amplified and checked for the BRAF V600E mutation by HRM (as described above).

The RPL27-PCR was processed through 45 cycles including 5 sec of denaturation at 95° C., a 7 sec annealing phase at 60° C. and an elongation phase at 72° C. for 9 sec. A 20 μA reaction consisted of 2 μA LightCycler FastStart DNA Master SYBR Green I (containing Taq DNA Polymerase, reaction buffer, dNTP mix (with dUTP instead of dTTP) and 10 mmol/l MgCl₂), additional 1.6 μl MgCl₂, 0.5 μmol/l of each primer, and 2 μl of template. The following primers were used:

RPL27-F:
(SEQ ID No. 23)
5′-ATCGCCAAGAGATCAAAGATAA-3′
and
RPL27-R:
(SEQ ID No. 24)
5′-TCTGAAGACATCCTTATTGACG-3′.

Both, RPL27 and TG mRNA could be amplified in at least 80% of samples providing at least 1 ml of material (++) (FIG. 6). Moreover, a screening for BRAF mutations by HRM was possible in all samples providing at least 1 ml of material (FIG. 7).

### 6. SELECTION OF miRNA SEQUENCES BY A NEXT GENERATION SEQUENCING (NGS) APPROACH IN A SET OF 25 FTA (BENIGN) AND 25 FTC (MALIGNANT)

By means of Next Generation Sequencing (NGS) new miRNA markers for the discrimination of benign and malignant thyroid tumors were identified. The NGS approach allows the identification and quantification of all the specific miRNA isoforms being expressed and belonging to a miRNA family resulting in much more specific miRNA markers for the discrimination of benign and malignant thyroid FNA samples compared to the studies published so far.

In detail, Illumina hiScan miRNA sequencing was performed to identify miRNA sequences present in the samples library constructed from 25 FTA (benign) and 25 FTC (malignant) samples. Samples were multiplexed in groups of 10 per one flow cell lane so an average of ˜200 Mbases was read per sample in total. Samples were de-multiplexed with Illumina CASAVA software hence for each sample fastq file FastQC quality control could be performed. Adapters observed in 51 bp reads were cut with cutadapt assuming length of miRNA in the range of 15-27 bp.

To perform further analysis of miRNA expression, Reads per Million (RPM) normalization was performed. Direct comparison of miRNA sequences cannot be performed since the number of reads obtained for each sample are different. RPM normalization was performed according to this formula:

$$RPM = \frac{{R}_{miR}}{{R}_{all}} * {10}^{6}$$

where
Rₘᵢᵣ—number of reads mapped to particular miR sequence,
Rₐₗₗ—total number of reads mapped,
RPM—normalized miRNA expression value.

Normalized RPM values were used for the miRNA expression calculations.

First, a decision tree based method was used to calculate the best classifier for the differentiation of benign and malignant samples with a minimal number of miRNA isoforms, each with a minimal fold change=2 between benign and malignant samples. The decision tree, the used miRNA isoform sequences and the classification of the 50 samples based on this decision tree is shown in FIG. 8.

Furthermore, hit lists of best differentiating and highly specific miRNA isoform sequences were calculated. These miRNA will be quantified in addition to the classifier sequences to improve discrimination between benign and malignant samples. MiRNA isoform sequences up-regulated in FTA in comparison to FTC are shown in Table 7, while miRNA isoform sequences up-regulated in FTC in comparison to FTA are shown in Table 8.

TABLE 7
miRNA isoform sequences up-regulated in benign tumor samples

| sequence | ID | median_FTC | median_FTA | fold change | t-test |
|---|---|---|---|---|---|
| TGGAAGACTAGTGATTTTGTTGT | 30 | 70 | 1202 | 0.058 | 0.00636 |
| TTCCCTTTGTCATCCTATGCCT | 31 | 163 | 996 | 0.163 | 0.01858 |
| TGGAAGACTAGTGATTTTGTTGTT | 32 | 220 | 1058 | 0.207 | 0.00722 |
| AAACCGTTACCATTACTGAGTTT | 33 | 260 | 801 | 0.324 | 0.00412 |
| TGTAAACATCCTCGACTGGA | 34 | 6983 | 19898 | 0.351 | 0.00032 |

TABLE 8
miRNA isoform sequences up-regulated in malignant tumor samples

| sequence | ID | median_FTC | median_FA | fold change | t-test |
|---|---|---|---|---|---|
| TGAGAACTGAATTCCATAGGCTG | 35 | 1167.8 | 548.2 | 2.130 | 0.03717 |
| ACCGGGTGCTGTAGGCTT | 36 | 1075.8 | 502.9 | 2.139 | 0.00484 |
| GAGAAAGCTCACAAGAACTG | 37 | 578.4 | 270.1 | 2.142 | 0.08515 |
| CCTGTCTGAGCGTCGCT | 38 | 6480.4 | 2728.2 | 2.375 | 0.01415 |
| TGAGAACTGAATTCCATAGGCTGT | 39 | 2426.8 | 959.8 | 2.528 | 0.07120 |

Moreover, hit lists of best differentiating miRNA seed sequences representing miRNA families were calculated. These miRNA families will be quantified in addition to the classifier sequences and the miRNA isoforms to further improve discrimination between benign and malignant samples. MiRNA seed sequences up-regulated in FTA in comparison to FTC are shown in Table 9, while miRNA seed sequences up-regulated in FTC in comparison to FTA are shown in Table 10.

TABLE 9
miRNA seed sequences up-regulated in benign tumor
samples

| | | median_ | median_ | fold | |
|---|---|---|---|---|---|
| sequence | ID | FTC | FTA | change | t-test |
| TGGAAGAC | 43 | 352 | 2967 | 0.119 | 0.00540 |
| TTCCCTTT | 44 | 375 | 1603 | 0.234 | 0.03649 |
| GTCCAGTT | 45 | 351 | 825 | 0.425 | 0.01074 |
| AACCCGTA | 46 | 2808 | 5661 | 0.496 | 0.00028 |
| AAACCGTT | 47 | 1835 | 4137 | 0.444 | 0.00454 |
| TCCTGTAC | 48 | 3520 | 7075 | 0.498 | 0.00626 |

TABLE 10
miRNA seed sequences up-regulated in malignant
tumor samples

| | | median_ | median_ | fold | |
|---|---|---|---|---|---|
| sequence | ID | FTC | FA | change | t-test |
| CCTGTCTG | 40 | 6914.3 | 2873.1 | 2.407 | 0.01436 |
| GAGAAAGC | 41 | 1888.2 | 899.1 | 2.100 | 0.08766 |
| ATGTTTAG | 42 | 747.2 | 328.4 | 2.275 | 0.00400 |

What is claimed is:

1. A method for distinguishing malignant from benign tumor samples of the thyroid, by
a.) performing a RNA extraction in an air dried and/or fixed fine needle aspiration biopsy (FNAB) sample,
b.) analyzing differential expression of miRNA and/or the presence of gene-rearrangements in the isolated RNA, wherein the presence of a gene-rearrangement and/or the differential expression of miRNA is indicative for a malignant tumor.

2. A method according to claim 1 wherein in step b.) the analyzed miRNA comprising the following nucleic acid sequences:
(SEQ ID No. 25)
TCGAGGAGCTCACAGTCTAGTAA,
(SEQ ID No. 26)
AATGTTTAGACGGGCTC,
(SEQ ID No. 27)
TACCCTGTAGAACCGAATTT,
(SEQ ID No. 28)
TCCTGTACTGAGCTGCCCCGAGA,
and/or
(SEQ ID No. 29)
AACATTCAACGCTGTCGGTGAA
or complementary sequences are detected.

3. A method according to claim 2, detecting at least one of the following miRNA isoforms comprising a nucleic acid sequence according to SEQ ID No. 30 to 39 or a complementary sequence and/or at least one miRNA comprising one of the following miRNA seed sequences according to SEQ ID No. 40 to 48 or a complementary sequence.

4. A method according to claim 1, wherein in step b.) rearrangements of RET/PTC and/or PAX8/PPARG (paired box 8/peroxisome proliferator-activated receptor gamma) are analyzed.

5. A method according to claim 1, wherein step b.) is carried out by RT-PCR or pyrosequencing.

6. A method according to claim 1, wherein in step a.) mRNA, miRNA and DNA are extracted simultaneously from the fine needle aspiration biopsy tumor sample.

7. A method according to claim 6, analyzing additionally point mutations in DNA and/or miRNA wherein the presence of a point mutation in the isolated DNA and/or miRNA is indicative for a malignant tumor.

8. A method according to claim 7 wherein point mutations in DNA encoding BRAF, N-, K-, and/or HRAS are analyzed.

9. A method according to claim 1, wherein the presence of RET/PTC and/or PAX8/PPARG rearrangements classifies the tumor as malignant.

10. A method according to claim 1, performing RNA isolation after cytological fixation and staining of the air dried and/or fixed fine needle aspiration biopsy (FNAB) sample.

11. A kit comprising at least one of the following:
i. Buffer for nucleic acid extraction,
ii. Primer, buffers and RNA-dependent-DNA-polymerase for Reverse transcription,
iii. Primers, buffers, dNTP mix, and a DNA-Polymerase for PCR on cDNA, or
i. Buffer for nucleic acid extraction,
ii. Reagents for labeling RNA and/or DNA,
iii. Hybridization buffers,
iv. Washing buffers,
for performing the method according to claim 1.

12. miRNA comprising a nucleic acid sequence selected from SEQ ID No. 25 to 48 as a marker for distinguishing malignant from benign tumor samples of the thyroid.

13. miRNA according to claim 12 selected from SEQ ID No. 25 to 29.


SEQUENCE LISTING (48 sequences)

SEQ ID NO: 1 - DNA, 21 bp, Artificial Sequence
  Primer
cctgctggctccaccttgttt

SEQ ID NO: 2 - DNA, 24 bp, Artificial Sequence
  Primer
ccttgttctgagcctcccatcgtt

SEQ ID NO: 3 - RNA, 23 nt, Homo sapiens
agcuacauugucugcuggguuuc

SEQ ID NO: 4 - DNA, 23 bp, Artificial Sequence
  Primer
tcataatgcttgctctgatagga

SEQ ID NO: 5 - DNA, 22 bp, Artificial Sequence
  Primer
ggccaaaaatttaatcagtgga

SEQ ID NO: 6 - DNA, 20 bp, Artificial Sequence
  Primer
tcaacctccctatggacagc

SEQ ID NO: 7 - DNA, 19 bp, Artificial Sequence
  Primer
ggagtaggtggagcccagg

SEQ ID NO: 8 - DNA, 20 bp, Artificial Sequence
  Primer
cctctcgactcaccagacct

SEQ ID NO: 9 - DNA, 23 bp, Artificial Sequence
  Primer
gcccttcaatgcctttccccatg

SEQ ID NO: 10 - DNA, 20 bp, Artificial Sequence
  Primer
agcggacagggcagctatgc

SEQ ID NO: 11 - DNA, 21 bp, Artificial Sequence
  Primer
ccaaagttggtgggccagaat

SEQ ID NO: 12 - DNA, 18 bp, Artificial Sequence
  Primer
catggttgacacagagat

SEQ ID NO: 13 - DNA, 24 bp, Artificial Sequence
  Primer
ggagacctacaaactgaagtgcaa

SEQ ID NO: 14 - DNA, 25 bp, Artificial Sequence
  Primer
cccttctcctagagtttttccaaga

SEQ ID NO: 15 - DNA, 20 bp, Artificial Sequence
  Primer
aaccgcgacctgcgcaaagc

SEQ ID NO: 16 - DNA, 24 bp, Artificial Sequence
  Primer
ccagtggttatcaagctccttaca

SEQ ID NO: 17 - DNA, 23 bp, Artificial Sequence
  Primer
gggaattcccactttggatcctc

SEQ ID NO: 18 - DNA, 20 bp, Artificial Sequence
  Primer
acccagcaccgacccccagg

SEQ ID NO: 19 - DNA, 22 bp, Artificial Sequence
  Primer
ggtgattttggtctagctacag

SEQ ID NO: 20 - DNA, 22 bp, Artificial Sequence
  Primer
ggccaaaaatttaatcagtgga

SEQ ID NO: 21 - DNA, 20 bp, Artificial Sequence
  Primer
aggcctgctgaaaatgactg

SEQ ID NO: 22 - DNA, 21 bp, Artificial Sequence
  Primer
gctgtatcgtcaaggcactct

SEQ ID NO: 23 - DNA, 22 bp, Artificial Sequence
  Primer
atcgccaagagatcaaagataa

SEQ ID NO: 24 - DNA, 22 bp, Artificial Sequence
  Primer
tctgaagacatccttattgacg

SEQ ID NO: 25 - DNA, 23 bp, Homo sapiens
tcgaggagctcacagtctagtaa

SEQ ID NO: 26 - DNA, 17 bp, Homo sapiens
aatgtttagacgggctc

SEQ ID NO: 27 - DNA, 20 bp, Homo sapiens
taccctgtagaaccgaattt

SEQ ID NO: 28 - DNA, 23 bp, Homo sapiens
tcctgtactgagctgccccgaga

SEQ ID NO: 29 - DNA, 22 bp, Homo sapiens
aacattcaacgctgtcggtgaa

SEQ ID NO: 30 - DNA, 23 bp, Homo sapiens
tggaagactagtgattttgttgt

SEQ ID NO: 31 - DNA, 22 bp, Homo sapiens
ttccctttgtcatcctatgcct

SEQ ID NO: 32 - DNA, 24 bp, Homo sapiens
tggaagactagtgattttgttgtt

SEQ ID NO: 33 - DNA, 23 bp, Homo sapiens
aaaccgttaccattactgagttt

SEQ ID NO: 34 - DNA, 20 bp, Homo sapiens
tgtaaacatcctcgactgga

SEQ ID NO: 35 - DNA, 23 bp, Homo sapiens
tgagaactgaattccataggctg

SEQ ID NO: 36 - DNA, 18 bp, Homo sapiens
accgggtgctgtaggctt

SEQ ID NO: 37 - DNA, 20 bp, Homo sapiens
gagaaagctcacaagaactg

SEQ ID NO: 38 - DNA, 17 bp, Homo sapiens
cctgtctgagcgtcgct

SEQ ID NO: 39 - DNA, 24 bp, Homo sapiens
tgagaactgaattccataggctgt

SEQ ID NO: 40 - DNA, 8 bp, Homo sapiens
cctgtctg

SEQ ID NO: 41 - DNA, 8 bp, Homo sapiens
gagaaagc

SEQ ID NO: 42 - DNA, 8 bp, Homo sapiens
atgtttag

SEQ ID NO: 43 - DNA, 8 bp, Homo sapiens
tggaagac

SEQ ID NO: 44 - DNA, 8 bp, Artificial Sequence
  miRNA
ttcccttt

SEQ ID NO: 45 - DNA, 8 bp, Homo sapiens
gtccagtt

SEQ ID NO: 46 - DNA, 8 bp, Homo sapiens
aacccgta

SEQ ID NO: 47 - DNA, 8 bp, Homo sapiens
aacccgta

SEQ ID NO: 48 - DNA, 8 bp, Homo sapiens
tcctgtac